(19) United States
(12) Patent Application Publication
(10) Pub. No.: US 2009/0149796 A1
(43) Pub. Date: Jun. 11, 2009

(54) Use of Iontophoresis and Ultrasound to Deliver Melanin or Other Chromophores for Laser Hair Removal

(76) Inventors: Dennis R. Jones, Chesapeake, VA (US); Evan D. Parker, Virginia Beach, VA (US)

Correspondence Address: Guerry L. Grune, 784 Villier Court, Virginia Beach, VA, 23452, US

(21) Appl. No.: 11/951,720

(22) Filed: Dec. 6, 2007

(51) Int. Cl. A61N 1/30; A61B 18/20

(52) U.S. Cl. 604/20; 606/9

(57) ABSTRACT

The method disclosed includes combining iontophoresis and ultrasound to deliver the appropriate chromophores to blonde, grey, white, vellus, and other non-pigmented or laser resistant hair follicles to facilitate photothermolysis hair removal using a laser, Intense Pulsed Light (IPL), a combination of laser and RF energy, or a combination of IPL and RF. This technique preferentially ultra-exposes the hair follicles to light energy which is absorbed preferentially by naturally and synthetic elements in and around the hair follicle. The result is focused photothermolysis damage resulting in death of the hair producing tissue. The combined use of iontophoresis and ultrasound with the appropriate chromophore and laser light allows for immediate and permanent removal of unwanted hair in selected locations on the body of mammals. This combination is so effective that the patient can be treated with single, or at least minimal, office visitations, thereby eliminating the need to continuously prepare and re-prepare the skin with melanin or other chromophores prior to multiple—in office—laser or IPL treatments for completing desired hair removal results. Additionally, this method discloses the use of iontophoresis together with High Intensity Focused Ultrasound (HIFU) and also Radio Frequency (RF) energy combined with High Intensity Focused Ultrasound (HIFU) to specifically provide necrotizing energy doses to target the regenerative zones of the hair and accomplish hair removal.

### FIELD OF INVENTION

The method disclosed includes the simultaneous combination of iontophoresis and ultrasound to deliver the appropriate chromophores to blonde, grey, white, vellus, and other non-pigmented or laser resistant hair follicles to facilitate photothermolysis hair removal using a laser, Intense Pulsed Light (IPL), a combination of laser and Radio Frequency (RF) energy, or a combination of IPL and RF. The electrical current used can be either pulsed or continuous.

### BACKGROUND OF INVENTION

Historically, there have been a number of options for the permanent removal of hair. Electrolysis has been the most commonly selected approach, in which an operator, usually an electrologist, attaches an electrode to each individual hair shaft, with the patient typically holding a second electrode. An electrical current is then passed through the hair shaft and the hair follicle through the papilla. This precisely directed current can induce permanent injury in the follicle and papilla, stopping the future production of the hair shaft.

Problems exist with the electrolysis technique, however. The success with which hair is permanently removed varies greatly from patient to patient. Moreover, the process is slow since each hair follicle must be individually treated, and there is some discomfort associated with the electric current.

Other approaches have been proposed that rely on flashlamps, instead of lasers. This has the advantage of a less expensive, reasonably portable light source, but flashlamps create their own control problems. It is difficult to deliver light from the flashlamp to the skin, so the lamp must be placed in proximity to the skin. The reflectors that surround the flashlamp and collect the light and direct it to the skin must be precisely built and calibrated. Any error can cause hot spots in the spatial energy distribution. This can lead to under-treatment in some areas and burning in others. Moreover, the bandwidth of the light from these flashlamps is broad, usually including visible light and stretching into the longer infrared wavelengths. These longer wavelengths are well absorbed by water that dominates the skin make-up. Thus, the light from these sources tends to penetrate the skin very poorly, which leads to the use of higher fluence levels to sufficiently treat deeper-lying hair producing structures with the concomitant risk of burning or damaging the skin.

The removal of hair using lasers is another approach that has found success. Numerous techniques have been taught in the prior art. Each, however, suffers from drawbacks such as poor ultimate success in stopping hair growth even after multiple treatments, excessive injury to the tissue surrounding the hair follicles and papilla, and excessively large and expensive laser systems.

One approach relies on a pulsed laser source and the use of an exogenous absorber. A commercially available hair dye solution is first applied to the skin containing the unwanted hair and allowed to migrate along the hair shafts and into the follicles. The skin is then irradiated with a spot size of approximately 0.5 centimeters using a Q-switched YAG laser, or other short pulsed laser system. The pulse durations used by the lasers tend to be short, 15 nsec for the Q-switched laser. It appears that the sub-microsecond pulse durations shocks the hair follicle, which stops hair production, but only for a limited time. After months, the follicle again begins to produce hair, requiring further treatments or other techniques to yield any lasting success.

Still other approaches use laser light delivery systems that inject light into only a single hair follicle at a time. These have the advantage of a reduced concern for damaging tissue between hair follicles but have many of the same disadvantages associated with the electrolysis. That is, each individual hair and hair follicle must be separately treated.

Long pulse ruby lasers have recently been used in hair removal. The high energy ruby lasers, however, are generally large, inefficient types of laser light generators, where very long pulses are generated.

Photothermolysis as a method of hair removal can be divided into two general methods. The first being essentially the application of laser energy selected to be absorbed preferentially by elements residing naturally within the proximity of the hair follicle (see Zaias—U.S. Pat. No. 5,059,192 and hereby incorporated by reference). The second being differentiated from the first by introduction of a foreign element to provide a radiation target with an absorption frequency different from the naturally occurring elements (see Tankovich—U.S. Pat. Nos. 5,226,907 and 5,425,728). In both cases, death of the hair follicle is caused by localized thermolysis and subsequent destruction of the living tissue. One objective of any method of laser depilation is to minimize the radiation absorption of the surrounding skin and thereby reduce collateral damage.

A limitation of the second method, that of applying laser light by which natural skin and hair elements are not energized, is the obvious restriction from potential use of many available laser light sources. Because melanin naturally occurring in the skin is generally darker in color than the surrounding skin cells and tends to absorb, to some extent, a broad range of frequencies of incident light energy, depilation methods which attempt to avoid excitation of such natural elements are greatly limited.

In one study (Walther T., et. al., 1998) selective photothermolysis on dark pigmented hair follicles treated with a normal-mode ruby laser (694-nm wavelength, pulse duration 0.5 ms, fluence 20 J/cm², spot size 2 mm) was evaluated. For 15 volunteers, four test areas each were selected. After shaving, the first area was irradiated once, the second twice, the third three times and the fourth served as control area. A punch biopsy was taken from each volunteer immediately after the first laser treatment. Four weeks after the last irradiation, no effect was found in six cases and little effect in another six cases (50-90% regrowth). Hair regrowth of less than 30% was observed in only three cases. Eight weeks after the last session, no effect was found in 11 cases, little effect in 2 cases (10%) and less than 30% regrowth in only 2 cases. Twelve weeks after the last treatment, no difference could be detected between the areas untreated and those treated by laser. The laser parameters applied in this study did not result in effective epilation of body hair. In some cases, a delay in growth of several weeks was noticed.

### DEFINITIONS

Iontophoresis is a non-invasive method of propelling high concentrations of a charged substance, normally medication or bioactive-agents, transdermally by repulsive electromotive force using a small electrical charge applied to an iontophoretic chamber containing a similarly charged active agent and its vehicle. To clarify, normally one or two chambers are filled with a solution containing an active ingredient and its solvent, termed the vehicle or the vehicle can be applied directly to the skin. The positively charged chamber, termed the anode will repel a positively charged chemical, and the negatively charged chamber, termed the cathode, will repel a negatively charged chemical into the skin. Iontophoresis is well classified for use in transdermal drug delivery. Unlike transdermal patches, this method relies on active transportation within an electric field. In the presence of an electric field electromigration and electro osmosis are the dominant forces in mass transport.

There are a number of factors that influence iontophoretic transport including skin pH, drug concentration and characteristics, ionic competition, molecular size, current, voltage, time applied and skin resistance. Studies suggest that comparable iontophoretic doses delivered at low currents over longer periods are more effective than those delivered by high currents over a short periods.

The isoelectric point of the skin is ˜4; therefore, under physiological conditions, with the surface of the skin also buffered at or near 7.4, the membrane has a net negative charge and electro osmotic flow is from anode (−) to cathode (+). The phenomenon of electro osmosis has been used as a means to augment the anodic delivery of (in particular) large, positively charged drugs, the transport numbers of which are often extremely small (and whose iontophoretic enhancement therefore depends heavily upon electro osmosis) and to promote the transdermal migration of uncharged, yet polar, molecules, the passive permeation of which is typically very small.

The application of a charge to the skin alters the skin's permeability increasing migration of the active ingredient into the epidermis. There are a number of pathways that the ingredients could take, but research suggests that the majority of drugs permeate the skin via appendageal pores, including hair follicles and sweat glands, although some delivery is via the paracellular channels and minimal quantities are transcellular.

Transport of lipophilic drug molecules is believed to be facilitated by its dissolution into the lipid matrix of the stratum corneum however hydrophilic drugs which are thought to permeate through the open pores or cutaneous appendages (hair follicle and sebaceous glands) only accounts for 0.1% of the total skin surface area.

The various mentioned methods for hair removal require repeated treatments and have not been particularly successful in permanently preventing the growth of hair. Therefore it is desirable to have a method and system to treat blonde, grey, white, vellus, laser resistant and residual hair such that a minimal amount of treatments are performed and such that the treatment does not damage adjacent skin, and permanently inhibits hair growth.

### DESCRIPTION OF PRIOR ART

U.S. Pat. No. 5,059,192 to Zaias, Nardo, unassigned, describes a method of hair depilation, comprising the steps of: a) aligning a laser light applicator substantially vertically over a hair follicle opening, said applicator having an aperture of sufficient area to surround a hair follicle and overlie its papilla;b) applying through said aperture to the hair follicle a pulse of laser energy of a wavelength which is readily absorbed by the melanin of the papilla and having a radiant exposure dose of sufficient energy and duration to damage its papilla so that hair regrowth is prevented and scarring of the surrounding skin is avoided.

U.S. Pat. No. 5,226,907 to Tankovich, Nikolai I., unassigned, describes a process for the permanent destruction of plurality of hairs growing on a section of human skin comprising the steps of applying to the hairs and skin section a contaminant having a high absorption of at least one frequency band of light which penetrates human skin, cleaning the skin leaving at least a portion of the hairs under the skin contaminated with the contaminant, illuminating the skin section with at least one frequency band of light, a significant portion of which is absorbed in the contaminant so as to permanently destroy the plurality of hair.

U.S. Pat. No. 5,425,728 to Tankovich, Nikolai I., unassigned, describes a process for the permanent removal, from a section of human skin, of a plurality of hairs growing in hair ducts from follicles at the bottom of the ducts and being nourished by skin tissue immediately surrounding the follicles essentially without damage to skin tissue except to the skin tissue immediately surrounding the follicles, comprising the steps of selecting a contaminant capable of infiltrating the hair ducts and having a high optical absorption of at least one frequency band of light which will penetrate the section of skin, applying the contaminant to the surface of skin in such a manner as to cause a quantity of the contaminant to infiltrate the hair ducts, at least partly removing the contaminant from the section of skin but leaving at least a portion of the contaminant in the hair ducts and illuminating the section of skin with the at least one frequency band of light, a significant portion of which penetrates the section of skin and is absorbed in a quantity of contaminant in the hair ducts, the quantity of contaminant in the ducts and the energy absorbed in it being sufficient to cause a reaction which destroys the hairs by causing death of the follicles or of the skin tissue feeding the follicles.

U.S. Pat. No. 7,276,058 to Altshuler, et. al., and assigned to Palomar Medical Technologies Inc., describes a method for treating at least a selected target region at depth of a patient's body while protecting non-targeted tissue by; (a) cooling the patient's skin surface to a temperature below normal body temperature for a selected duration; (b) selectively applying radiation to the patient's skin above the region at least one of before, during and after step (a); and (c) repeating at least one of steps (a) and (b) for a selected number of cycles. The temperature to which the patient's skin surface is cooled and the duration is sufficient to cool the region to a selected temperature below normal body temperature during at least cooling portions of cycles at or below the border zone of the dermis and hypodermis. Additionally, described is a method including stimulating said selected region during at least one of before, during and after at least one of steps (a) and (b) whereby stimulating is performed at least one of mechanically, acoustically and electrically.

U.S. Pat. No. 6,090,101 to Quon, et. al., and unassigned, describes a method for permanent hair removal from living human skin having unwanted growing hair by destroying undesired hair cells using the steps of: a) introducing a compound containing alkaline ions into the skin surface in the region of undesired hair; and b) heating the alkaline ions with radiant energy providing at least 1.0 joule/cm², whereby the alkaline ions attack and destroy hair cells in various stages of growth. The process includes alkali ions that are transported to the site of the hair cells to be destroyed by massaging the skin, using ultrasound or by a direct current electrical potential to which the alkali ions have been applied.

U.S. Pat. No. 7,250,047 to Anderson, et. al., and assigned to Lumenis Ltd., describes a device having a cavity with at least two electrodes having contact surfaces adapted to contact tissue collected therebetween, and the cavity further having a suction lumen in communication therewith. Also, an optical energy source adapted for transmitting optical energy to an outer surface of the tissue collected between the electrodes and an electrical energy source connected to the electrodes. In addition, an electromagnetic radiation may include, for example, therapeutic ultrasonic waves, therapeutic light treatment, therapeutic radio frequency (RF) treatment, etc. where two or more energy transmitting elements may be used.

U.S. Pat. No. 6,629,971 to McDaniel, David H., unassigned, describes a process for stimulating growth of hair exiting mammalian skin through a hair duct, the process comprising selecting at least one of a photoactive agent and a photo sensitizing agent, the agent having an average diameter enabling the agent to penetrate the hair duct, the agent having an electromagnetic radiation absorption characteristic enabling the agent to absorb at least a first wavelength of electromagnetic radiation from a skin-penetrating electromagnetic radiation source applying the agent to the hair and skin, forming an agent-tissue complex by a process comprising reacting the agent with cells in the hair, the skin, or both, and exposing the agent-tissue complex to incoherent, multi-frequency electromagnetic radiation comprising at least the first wavelength of electromagnetic radiation, whereby the agent-tissue complex absorbs the first wavelength of electromagnetic radiation.

U.S. Patent Publication No. 20020065513A1 to Richter, Jacob, and unassigned, describes a method of treating body tissue using the steps of: a) preparing a device capable of generating electrical current at a therapeutic voltage and amperage in response to ultrasonic vibrations; b) disposing the device in the vicinity of the tissue to be treated; and c) subjecting the device to ultrasonic vibrations in an amount and for a period of time sufficient for the device to generate electrical current at a therapeutic voltage and amperage. The device contains a housing with a medicament storage compartment; b) an oscillating member attached to the housing and communicating with the medicament storage compartment, the oscillating member adapted to oscillate in response to ultrasonic stimulation; c) a medicament port disposed on the housing and in fluid communication with the medicament storage compartment, the medicament port adapted to permit a medicament to be introduced into and contained in the medicament storage compartment and further adapted to selectively release the medicament from the medicament storage compartment in response to oscillations of oscillating member.

U.S. Patent Publication No. 20020147424A1 to Ostrow, et. al., and unassigned, describes a system for transdermal magnetic delivery of a substance in solution to an acceptor using at least one substance delivery means employing electroporation of the acceptor in the presence of a magnetic field, and also employing, in sequential fashion, a mode for active transport; and means for controlling at least one substance delivery means. The system is operable to provide combined electroporation of the acceptor and active transport of the substance in solution, in a controlled fashion, thereby delivering the substance to the acceptor. Additionally, the active transport mode comprises at least one of iontophoresis, electromagnetophoresis, sonophoresis, and photophoresis. The combination of iontophoresis, electroporation and magnetophoresis comprise the ionic drive in which the combination of an electric field and a magnetic field applied to molecules in a solution, activates the affected molecules for transport.

### SUMMARY OF THE INVENTION

It is an objective of the present disclosure to provide an improved method of permanent hair removal which employs iontophoresis and ultrasound or High Intensity Focused Ultrasound (HIFU) simultaneously or in conjunction with transdermal delivery of agents that may be delivered in pure form, in solution, as emulsions, within liposomes, within synthetic or natural microspheres, polymeric nanoparticles, microparticles, microcapsules, or other known micro encapsulation vehicles. Agents may include, without limitation, natural or synthetic melanin, aminolevulinic acid (ALA), hair dyes, vegetable dyes, food coloring, fabric dyes, tissue stains, shoe or leather dyes, and other plant products such as flavonols, cholorophyll, caroteniods, enzymes, etc.

Agents may be delivered in pure form, in solution, in emulsions, in liposomes, in synthetic or natural microspheres, polymeric nanoparticles, microparticles, microcapsules, or other known micro encapsulation and include a topical gel, lotion, liquid, cream or ointment composition that may also include melanin encapsulated liposomes. The agent is designed to attach to, or become physically incorporated into, the hair shaft, the hair follicle, the hair bulb, the hair duct and or papilla.

The ultrasound energy causes break up of the top most surface of the skin and allows the melanin encapsulated liposome composition to migrate into the hair follicle. This technique preferentially ultra-exposes the hair follicles to light energy which is absorbed preferentially by naturally occurring and synthetic elements in and surrounding the hair follicle. The result focuses photothermolysis damage resulting in death of the hair producing tissue. Melanin is one of the most common chromophores found in the hair follicle used for the selective photothermolysis of unwanted hair, and other chromophores such as ALA can be used also. Laser or other intense pulsed light between 100 and 3000 nm is appropriate for targeting melanin and ALA in the hair shaft.

Normal human hairs can be classified according to the 3 phases of their growth cycle: anagen, catagen, and telogen. Anagen hairs are in a growing phase, during which the matrix cells of the hair follicle undergo vigorous mitotic activity.

Toward the end of the anagen phase, the amount of pigment decreases at the base of the follicle, which expands to form a keratinized club. Then, the hair enters the catagen phase, a transitional phase in which mitotic activity decreases. The follicle separates from the dermal papilla and the capillary plexus and moves upward within its connective tissue sheath toward the epidermis. The resulting telogen hairs, or club hairs, are in a resting phase. These hairs have short, club-shaped roots that anchor them in the follicle. They lack root sheaths and show depigmentation of the proximal part of the shaft.

The hairs continue in this resting state until the follicle spontaneously reenters the anagen phase. At this point, the club hairs are forced out by growing hairs underneath them, and the cycle begins anew. The cycle is not synchronous throughout the scalp; patches of hair may be synchronized. The length of each phase of the cycle, as well as the length of the entire cycle, varies with the site and the age of the patient. In the scalp, for example, the average length of the anagen phase is 1000 days; that of the catagen phase, only a few days; and that of the telogen phase, 100 days. Of the 100,000 hairs on the average scalp, 10-15% are in the catagen or telogen phase at any time. Most hair follicles are in the anagen stage at any given point in time.

The combined use of melanin encapsulated liposomes or other chromophores in conjunction with iontophoresis and ultrasound or iontophoresis and HIFU followed by a treatment using a laser, Intense Pulsed Light (IPL), a combination of laser and Radio Frequency (RF) energy, or a combination of IPL and RF allows for immediate and permanent removal of unwanted hair in selected locations on the body of mammals. This combination is so effective that the patient can be treated with a single or at least minimal office visitation thereby eliminating the need to continuously prepare and re-prepare the skin with melanin or other chromophores prior to multiple—in office—laser treatments for completing desired hair removal results primarily in removing hairs that are primarily in the anagen phase.

It is another object of the disclosure to provide a composition where the liposomes have a size range of 1 nanometer (nm) to 500 microns. However, a diameter between 1 nanometer and 10 microns is preferred.

It is another object of the disclosure to provide a method of hair removal which increases the quantity of melanin or other chromophores surrounding the hair follicle to create an enhanced target for incident light energy and improve the preferential absorption in the proximity of the hair follicle.

It is another object of the disclosure to provide an improved method of laser hair removal which uses laser, IPL, a combination of laser and RF energy, a combination of IPL and RF or other light energy of a wavelengths or multiple wavelengths which are absorbed by naturally occurring or synthetic elements such as melanin in the skin or hair.

It is also an object of the disclosure to provide a method of destroying skin cancer cells and treating acne by photothermolysis by selective delivery of melanin using liposomes to create an enhanced target for subsequent application of light energy, which minimizes required irradiation and thereby minimizes the damage to the surrounding skin.

It is a further object of the disclosure that a variety light energy sources, both coherent and incoherent, including such lasers as ruby red and Nd-YAG, alexandrite, and diode lasers, or any other light source providing an effective frequency and intensity.

It is also another object of the disclosure that the light energy should be in the wavelength range of 100 to 3000 nm.

It is also another object of the disclosure that the pulse length is from about 1 nanoseconds to about 1000 milliseconds.

For the ultrasound application, High Intensity Focused Ultrasound (HIFU) can be provided in an innovative and cost-effective hair-removal appliance for cosmetic use.

The ultrasonic energy integrates low-cost piezoelectric crystals and a pioneering method for the production of HIFU.

This proprietary technology (patent pending) is utilized to specifically target necrotizing energy doses into the regenerative zones of the hair. Applisonix's ultrasonic design is expected to make its use familiar, friendly and safe for both professionals and common domestic users.

The technology is expected to be significantly safer than laser/IPL, because the energy is applied directly into the hair shaft and not irradiated over large skin surfaces (such as in laser and IPL treatments). Likewise, counter to the technological limitations of laser/IPL, this ultrasound technology is effective regardless of hair color or skin tone.

It is also an object of the disclosure to provide a more effective method of destroying regenerative zones of the hair by combining RF energy with HIFU to provide a more effective treatment.

It is also an object of the disclosure to provide a more effective method of delivering melanin by combining the delivery of melanin or other agents by Iontophoresis together with HIFU to provide a more effective treatment.

The combined use of iontophoresis and ultrasound, the appropriate chromophore and laser light allows for immediate and permanent removal of unwanted hair in selected locations on the body of mammals. This combination is so effective that the patient can be treated with a single or at least minimal office visitations thereby eliminating the need to continuously prepare and re-prepare the skin with melanin or other chromophores prior to multiple—in office—laser or IPL treatments for completing desired hair removal results.

Additionally, this method discloses the use of Radio Frequency (RF) energy combined with High Intensity Focused Ultrasound (HIFU) to specifically target necrotizing energy doses into the regenerative zones of the hair and accomplish hair removal.

### BRIEF DESCRIPTION OF THE DRAWINGS

FIG. 1 is an illustration that indicates how the method of combining iontophoresis and ultrasound is effective in driving particles into and around the hair shaft, follicle and papilla to provide a target sufficient for effective laser hair removal.

### DETAILED DESCRIPTION OF THE DRAWINGS

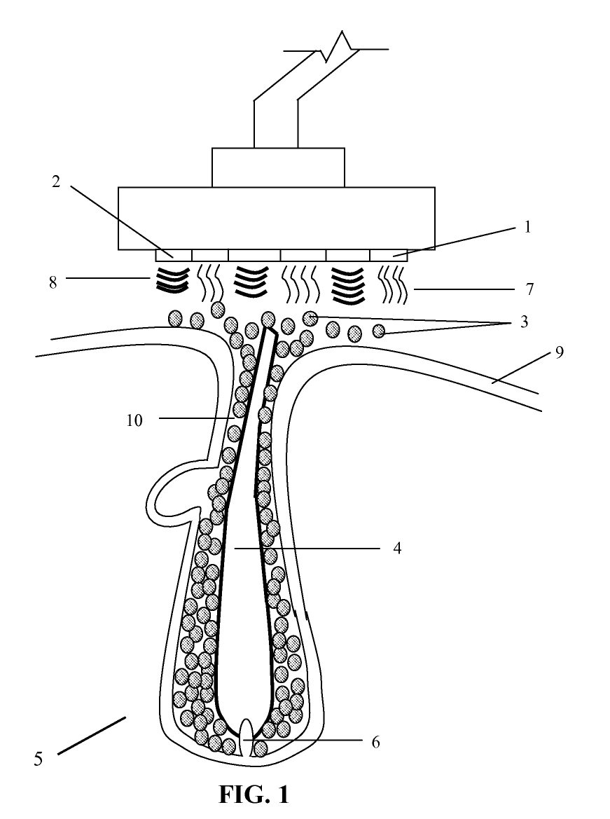

FIG. 1 is an illustration how the method of combining iontophoresis [1] and an ultrasound or HIFU generator [2] is effective in driving target agents [3] into and around the hair shaft [4], hair follicle [5] and papilla [6] to provide a sufficient amount of target agents [3] for effective laser hair removal (not shown). The target agents [3] are topically applied to the epidermis [9] near the hair shaft [4] to be removed.

An electrical current [7] produced by iontophoresis [1] as well as ultrasound waves [8] produced by an ultrasound generator [2] are generated simultaneously working in conjunction to assist with carrying the target agents [3] from the epidermis [9] into the hair duct [10] and hair follicle [5] surrounding the hair shaft [4]. The target agents [3] may be any of or a combination of natural or synthetically derived melanin, ALA, hair dyes, vegetable dyes, food coloring, fabric dyes, tissue stains, shoe leather dyes, and other plant derived products including flavanols, chlorophyll, carotenoids, and enzymes. Additionally the target agents [3] may be a topical gel, lotion, liquid, cream or ointment formulation and also comprising melanin encapsulated liposomes. Movement of the target agents [3] into the hair follicle [5] causes the hair shaft [4] to become physically incorporated with the target agents [3].

Selection of the target agents [3] to be used is dependent on the type and laser frequency, or photothermolysis, being used for hair removal. In a preferred mode the laser frequency matches the absorptive frequency of the target agent [3]. As the hair follicle [5] with the target agent [3] is exposed to the laser frequency, the hair shaft [4] and target agents [3] molecules begin to vibrate at their natural frequencies, generating heat and destroying the hair shaft [4], papilla [6], other structures in the follicle, and target agents [3].

1. A method of providing permanent hair removal employing a combination of;
iontophoresis and ultrasound, Intense Pulsed Light (IPL) and laser or equivalent light energy, wherein said combination includes using iontophoresis and ultrasound or High Intensity Focused Ultrasound (HIFU) simultaneously for transdermal delivery to skin with hair producing tissue, also providing natural or synthetically derived melanin, aminolevulinic acid (ALA), hair dyes, vegetable dyes, food coloring, fabric dyes, tissue stains, shoe leather dyes, and other plant derived products including in combination or individually; flavanols, chlorophyll, carotenoids, and enzymes by using a topical gel, lotion, liquid, cream or ointment formulation, wherein said method also comprises melanin encapsulated liposomes or chromophores other than melanin using ultrasound for causing break up of the top most surface of said skin, allowing said melanin encapsulated liposomes or chromophores other than melanin to migrate into said skin directed toward hair follicles such that said liposomes or chromophores other than melanin may reside in or become physically incorporated into, the hair shaft, the hair follicles, the hair bulb, or the hair duct so that using IPL, laser, a combination of laser and Radio Frequency (RF), a combination of IPL and RF or equivalent light energy, wherein electrical current is either pulsed or continuous resulting in preferential ultra-exposure of said hair follicles providing optimal focus of photothermolysis thereby facilitating destruction of hair producing tissue.

2. The method of claim 1, wherein said permanent hair removal is provided by one or more patient treatments.

3. The method of claim 1, wherein melanin in the hair follicle is the chromophore for the selective photothermolysis of unwanted hair.

4. The method of claim 1, wherein a laser or other intense pulsed light between 100 to 3000 nm is used for targeting melanin in a hair follicle shaft.

5. The method of claim 1, wherein permanent hair removal employing a combination of; iontophoresis, ultrasound or HIFU, and laser or equivalent light energy is required for eliminating the need for continuously preparing and re-preparing the skin with melanin or other chromophores prior to multiple in-office laser treatments for completion of desired permanent hair removal.

6. The method of claim 1, comprising the step of exposing said skin to at least one enzyme prior to exposure to ultrasound and iontophoresis.

7. The method of claim 1, wherein removal of hair is primarily black, blond, white, grey, vellus, laser resistant, or residual hair.

8. The method of claim 1, wherein the pulse length for laser light is from about 1 nanosecond to about 1 second.

9. The method of claim 8, wherein said pulse length is from about 1 nanosecond to about 1000 milliseconds.

10. The method of claim 1, wherein the laser or equivalent light energy fluence received at said hair producing tissue is no greater than 300 J/cm².

11. The method according to claim 10, wherein the energy fluence received at hair producing tissue is either between about 1×10⁻⁶ J/cm² to 1 J/cm² or is greater than 1 J/cm².

12. The method of claim 11, further comprising cooling said hair producing tissue to maintain a temperature of said tissue below a threshold for thermal injury and further comprising the step of maintaining the temperature of said tissue at or below 38 degrees Celsius.

13. A set of devices that provide for permanent hair removal employing a combination of, iontophoresis and ultrasound, Intense Pulsed Light (IPL) and lasers or equivalent light energy, wherein said combination includes using iontophoresis and ultrasound or High Intensity Focused Ultrasound (HIFU) simultaneously for transdermal delivery to skin with hair producing tissue and also providing natural or synthetically derived melanin, aminolevulinic acid (ALA), hair dyes, vegetable dyes, food coloring, fabric dyes, tissue stains, shoe leather dyes, and other plant derived products including in combination or individually; flavanols, chlorophyll, carotenoids, and enzymes by using a topical gel, lotion, liquid, cream or ointment formulation, wherein said method also comprises using melanin encapsulated liposomes or chromophores other than melanin using ultrasound for causing break up of the top most surface of said skin, allowing said melanin encapsulated liposomes or chromophores other than melanin to migrate into said skin directed toward hair follicles such that said liposomes or chromophores other than melanin may reside in or become physically incorporated into, the hair shaft, the hair follicles, the hair bulb, or the hair duct so that using IPL, laser, a combination of laser and Radio Frequency (RF), a combination of IPL and RF or equivalent light energy, wherein electrical current is either pulsed or continuous resulting in preferential ultra-exposure of said hair follicles providing optimal focus of photothermolysis thereby facilitating destruction of hair producing tissue.

14. The devices of claim 13, wherein said permanent hair removal is provided by one or more patient treatments.

15. The devices of claim 13, wherein melanin in the hair follicle is the chromophore for the selective photothermolysis of unwanted hair.

16. The devices of claim 13, wherein a laser or other intense pulsed light between 100 to 3000 nm is used for targeting melanin in a hair follicle shaft.

17. The devices of claim 13, wherein permanent hair removal employing a combination of; iontophoresis, ultrasound or HIFU, and laser or equivalent light energy is required to eliminate the need for continuously preparing and re-preparing the skin with melanin or other chromophores prior to multiple in-office laser treatments for completion of desired permanent hair removal and wherein said skin is exposed to at least one enzyme prior to exposure to ultrasound and iontophoresis.

18. The devices of claim 13, wherein removal of hair is primarily black, blond, white, grey, vellus, laser resistant, or residual hair.

19. The devices of claim 13, wherein the pulse length for laser light is from about 1 nanosecond to about 1 second, or said pulse length is from about 1 nanosecond to about 1000 milliseconds and wherein said laser or equivalent light energy fluence received at said hair producing tissue is no greater than 300 J/cm².

20. The devices of claim 19, wherein the energy fluence received at hair producing tissue is either between about 1×10⁻⁶ J/cm² to 1 J/cm² or is greater than 1 J/cm² and wherein cooling said hair producing tissue to maintain a temperature of said tissue below a threshold for thermal injury and further comprising the step of maintaining the temperature of said tissue at or below 38 degrees Celsius is required.
